(19) United States
(12) Patent Application Publication
(10) Pub. No.: US 2005/0153375 A1
(43) Pub. Date: Jul. 14, 2005

(54) CEA/NCA-based differentiation cancer therapy

(76) Inventors: Clifford P. Stanners, Ontario (CA); Christian Ilantzis, Longueuil (CA); Cosme Ordonez-Garcia, Montreal (CA); Maryam Taheri, Pierrefonds (CA); Robert A. Screaton, La Jolla, CA (US); Abraham Fuks, Montreal (CA); H.U. Saragovi, Montreal (CA)

Correspondence Address: Ralph A. Dowell of DOWELL & DOWELL P.C., 2111 Eisenhower Ave., Suite 406, Alexandria, VA, 22314, US

(21) Appl. No.: 11/041,199

(22) Filed: Jan. 25, 2005

Related U.S. Application Data
(63) Continuation of application No. 09/637,530, filed on Aug. 11, 2000, now abandoned.
Application No. 09/637,530 is a continuation of application No. PCT/CA99/00119, filed on Feb. 11, 1999.

(30) Foreign Application Priority Data
Feb. 12, 1998 (CA) 2,224,129

(51) Int. Cl. G01N 33/574; A61K 38/17; C07K 14/47

(52) U.S. Cl. 435/7.23; 514/2; 530/350

(57) ABSTRACT

The present invention relates to a novel cancer therapy based on interference with the function or on downregulation of overproduced CEA/NCA, which plays an instrumental role in tumorigenesis and malignant progression through its differentiation-blocking activity. More precisely, there is provided three short amino acid sequence subdomains in the N domain of CEA and NCA that, when applied as peptides, peptide mimetics or anti-sudomain monoclonal antibodies to malignant tumors overproducing CEA/NCA, induce them to differentiate, thereby inhibiting their ability to grow and increasing the efficacy of other modes of treatment. Four other means of releasing the CEA/NCA-imposed differentiation block are also provided. The enhanced differentiation status of cancers induced by these CEA/NCA-based novel modes of treatment is expected to increase the efficacy of virtually any other mode of treatment by enhancing the bystander effect, whereby more differentiated cancer cells normalize the behaviour of adjacent less differentiated cancer cells.

### CROSS-REFERENCE TO RELATED APPLICATIONS

This application is a continuation of U.S. patent application Ser. No. 09/637,530, filed Aug. 11, 2000, which is a continuation application of PCT/CA99/00119, filed Feb. 11, 1999, which is an international filing of Canadian Patent Application No. 2,224,129, filed Feb. 12, 1998, all of which are incorporated herein by reference.

### BACKGROUND OF THE INVENTION

(a) Field of the Invention

The invention relates to a novel cancer therapy based on the direct or indirect downregulation of endogenous CEA/NCA which plays an instrumental role in malignant progression through its differentiation-blocking activity on normal cells.

(b) Description of Prior Art

The long-term cure rates for cancers at many sites treated by present means, such as surgery, radiation and chemotherapy, are often unacceptably low. Due to the common presence of metastases derived from the primary tumor, it is impossible to treat most cancers effectively with surgery and radiation alone. Systemic chemotherapy is effective in some cases but is often too toxic to permit the use of the doses required for cure. Novel treatments based on molecular differences between cancer and normal cells are required. Such treatments would likely be non-toxic and, since they would be based on different principles from the commonly used treatments, would be expected to be synergistic with them, giving more effective combined treatment.

Tumor cells at many sites, including colon, breast, lung, cervix, ovary, stomach, bladder, pancreas and esophagus express large amounts of carcinoembryonic antigen (CEA) and/or the closely related family member, NCA, on their surfaces. The expression of these glycoproteins, especially CEA, in normal cells is very limited. This represents the basis for the wide clinical use of CEA as a blood tumor marker. Since the majority of human cancers show up-regulation of CEA/NCA, any therapy based on this fact has potential application to an immense number of cancer patients. This upregulation in so many types of cancer led us to suggest that CEA and NCA could actually contribute directly to tumorigenicity. We first showed that CEA (Benchimol et al, Cell 57:327-334, 1989) and NCA (Zhou et al, Cell Growth Differ. 1:209-215, 1990) function as intercellular adhesion molecules. Although CEA had been previously considered as an inert marker of tumorigenicity, we suggested that inappropriate CEA/NCA expression in cells still capable of proliferation could cause a distortion of tissue architecture (which is determined by adhesion molecules) and an inhibition of terminal differentiation that normally removes cells from the pool of cells with potential to proliferate, thus contributing directly to malignant progression.

In agreement with this hypothesis, we have shown that CEA and NCA expression in transfected myoblasts can inhibit terminal myogenic differentiation and promote tumorigenicity. Peptides representing the adhesion domains of CEA can release the myogenic differentiation block in CEA-transfected myoblasts, indicating the necessity of CEA-CEA interactions for the inhibition of terminal differentiation.

In the present invention, the inhibition of terminal differentiation by CEA/NCA over-expression has been demonstrated to apply to the adipogenic differentiation of mouse fibroblasts, to the neuronal differentiation of mouse embryonal carcinoma cells and to the differentiation and polarization of human colonocytes. CEA/NCA over-expression has also been shown to distort tissue architecture and to inhibit anoikis (apoptosis of anchorage-free cells). The inhibition of differentiation depends on both interactions between the external domains of CEA and on the presence of a CEA-determined glycophosphatidyl-inositol (GPI) membrane anchor. Novel treatments based on reversal of the carcinogenetic effects of CEA/NCA would be highly desirable since they would be precisely targeted to tumor cells expressing these molecules and should therefore be applicable to a large proportion of human cancers. Reversal can be achieved by interference with certain subdomains of CEA and NCA that are required for the differentiation-blocking activity, by downregulating the cellular production of these molecules and by three other means, as delineated below.

It would be highly desirable to provide a novel cancer therapy based on the direct or indirect downregulation of endogenous CEA/NCA which plays an instrumental role in malignant progression, through its differentiation-blocking activity on normal cells.

### SUMMARY OF THE INVENTION

One aim of the present invention is to provide a novel cancer therapy based on interference with the differentiation-blocking activity of CEA/NCA or on downregulating the production of CEA/NCA, which we show plays an important role in malignant progression. This treatment based on this property of CEA/NCA is precisely targeted to tumor cells expressing these molecules and is therefore non-toxic and applicable to a large proportion of human cancers.

In accordance with the present invention, there exists five (5) different routes for interference with the tumorigenic effects of CEA/NCA:
1. Antibodies raised against subdomains of CEA/NCA, small peptides and derived mimetics (organic structures that mimic peptides) which all interact with precise subdomains of CEA/NCA that are involved in the differentiation-blocking activity associated with malignant progression. The antibodies, peptides and derived mimetics when administered to cancer patients will restore differentiation and thus inhibit tumorigenicity.
2. Antisense cDNA constructs, ribozymes and oligonucleotides reduce the expression of CEA/NCA when administered directly or by gene therapy to a cancer patient, which restores differentiation and thus inhibits tumorigenicity.
3. Cell surface receptors involved in extracellular matrix (ECM) binding, integrins, are involved in the differentiation-blocking activity of CEA/NCA molecules. The particular integrins, including α₅β₁, and αᵥβ₃ have been identified. Inhibition of the changes that these molecules undergo as a result of CEA/NCA activity could release the CEA/NCA-imposed differentiation block. Certain monoclonal antibodies directed against these particular integrins have this effect and when administered to patients will restore differentiation and thus inhibit tumorigenicity.
4. “Shankless anchors”, consisting of the GPI anchor of CEA without the external domains, interfere with the differentiation-inhibiting activity of CEA/NCA and can be administered directly to a cancer patient. The “shankless anchors” inhibit the differentiation-blocking activity of the endogenous CEA/NCA molecules by competing with CEA/NCA for the elements of the molecular pathway required for the CEA/NCA effect.
5. Agents that interfere with the signaling process between CEA/NCA molecules activated by self association and the aforementioned integrins, thus interfering with the changes in the latter that inhibit differentiation, could release the CEA/NCA-imposed differentiation block.

All these routes lead to new useful clinical agents capable of removing tumor cells by inducing their terminal differentiation, thus effectively killing tumor cells by a mechanism distinct from that of cytotoxic drugs.

In accordance with the present invention there is provided an inhibiting CEA/NCA sequence, which comprises antisense mRNA sequences which hybridize to at least one domain of CEA/NCA selected from the group consisting of the cDNA sequences of CEA and NCA to reduce the expression of endogenous CEA/NCA when administered to a cancer patient.

In accordance with the present invention there is also provided inhibiting CEA/NCA nucleotide sequences, wherein the sequence is an antisense cDNA, an antisense oligonucleotide or an antisense ribozyme containing CEA/NCA antisense nucleotide sequences.

In accordance with the present invention there is also provided anti-CEA/NCA antibodies, which comprise antibodies raised against subdomains of CEA/NCA involved in the differentiation-blocking activity associated with tumorigenicity, wherein the subdomains are selected from the group consisting of the sequences G₃₀YSWYK, N₄₂RQII, Q₈₀ND and other sequences in the N terminal 107 amino acid domain, and sequences in the internal A3B3 domain of CEA.

In accordance with the present invention there is also provided peptides and peptide-derived mimetics, which comprise peptide and peptide-derived mimetics interacting with subdomains of CEA/NCA involved in the differentiation-blocking activity associated with tumorigenicity, wherein the subdomains are selected from the group consisting of the sequences G₃₀YSWYK, N₄₂RQII, Q₈₀ND and other sequences in the N-terminal 107 amino acid domain, and sequences in the internal A3B3 domain of CEA.

The present invention includes also combinations of peptides representing these subdomains in which the peptides are free or linked together with polyethylene glycol molecules.

In accordance with the present invention there is also provided a shankless anchor, which comprises a GPI anchor of CEA without the external peptide domains attached, wherein the GPI anchor interferes with downstream targets of endogenous CEA/NCA molecules to inhibit the differentiation-blocking activity of the endogenous CEA/NCA molecules.

In accordance with the present invention there is also provided a method to restore endogenous integrin function including integrins α₅β₁ and αᵥβ₃, which comprises the steps of:
a) administration of monoclonal antibodies that reverse the CEA/NCA-induced changes in integrin function; and
b) administration of peptides/mimetics that mimic the effect of the mabs;

thereby inhibiting the differentiation-blocking activity of the endogenous CEA/NCA molecules.

In accordance with the present invention there is also provided a drug screen assay utilizing CEA/NCA-expressing transfectants of rat L6 myoblasts to determine pharmaceutical agents which are capable of inhibiting the signaling process required for differentiation-blocking activity of the endogenous CEA/NCA molecules, which comprises the steps of:
a) screening for agents capable of releasing the myogenic differentiation block in rat L6 cells expressing CEA/NCA; and
b) screening for agents capable of restoring normal cellular and tissue architecture to human Caco-2 colonocytes aberrantly expressing high levels of CEA/NCA.

In accordance with the present invention there is also provided the use of the anti-CEA/NCA antibodies, the peptides and peptide-derived mimetics, the inhibiting CEA/NCA sequence, or the shankless anchor of the present invention, to enhance efficacy of other anti-cancer treatments by increasing the differentiation status of a tumor and by enhancing the bystander effect; whereby more differentiated tumor cells cause more adjacent autonomous tumor cells to behave more as non-malignant or normal cells. Other treatment modes will not be required to kill as many tumor cells in order to be efficacious.

In accordance with the present invention there is also provided anti-CEA/NCA antibodies, the peptides and peptide-derived mimetics, the inhibiting CEA/NCA sequence, or the shankless anchor of the present invention, to restore anoikis/apoptosis to levels of non-malignat or normal cells, thereby increasing the efficacy of all other cytotoxic chemotherapeutic drugs which depend on apoptosis for killing cells.

### BRIEF DESCRIPTION OF THE DRAWINGS

FIG. 1 illustrates the effect of CEA/NCA overexpression in normal epithelial cells of colonic crypts which contributes to the development of a malignant tumor. The different sites of action of the routes 1, 2, 3, 4 and 5 of the present invention as described above are indicated;

FIG. 2 illustrates that overexpression of NCA in human colorectal carcinoma cells, SW-1222, blocks the formation of glandular-like structures of polarized cells with central lumens in monolayer culture;

FIG. 3 (top) illustrates that everexpression of NCA blocks the formation in collagen gels of glandular spheroids consisting of radially arranged polarized colonocytes with central lumens;

FIG. 3 (bottom) illustrates that overexpression of CEA and NCA in human colorectal carcinoma cells, Caco-2, blocks their polarization in monolayer culture leading to tumor-like multilayered structures with circumferential expression of CEA;

FIG. 4 illustrates that dome formation, due to vectorial transport of solvent from apical to basolateral surfaces of colonocytes and an indicator of polarization, is strongly inhibited by over-expression of CEA/NCA in Caco-2 colonocytes;

FIG. 5 illustrates that overexpression of NCA in SW-1222 cells causes loss of colonic glandular crypt formation with polarized cells facing a central lumen in a tissue architecture assay in vivo;

FIG. 6 illustrates that NCA overexpression inhibits anoikis (apoptosis) of SW-1222 cells cultured in suspension;

FIG. 7 illustrates that down-regulation of CEA in SW-1222 cells results in more normal, less tumorigenic cellular characteristics;

FIG. 8 illustrates the quantitative evidence that down-regulation of CEA in SW-1222 cells results in more glandular spheroids with recognizable central lumens;

FIG. 9 illustrates the subdomains in the NH2-terminal N domain of CEA that are required for intercellular adhesion and for the myogenic differentiation block. The quantitative effects of mutations in these subdomains on CEA-mediated adhesion and on CEA-mediated myogenic differentiation block are also shown;

FIG. 10 illustrates photomicrographs of L6 transfectants indicated in FIG. 9, showing the extent of myogenic differentiation in some of the subdomain mutants;

FIG. 11 illustrates the effects of the addition of small cyclic peptides, that include the amino acid sequences indicated in the three N domain subdomains, on the myogenic differentiation of rat L6 myoblasts. Myogenic differentiation is indicated by positive labelling with fluorescent anti-myosin antibody. CEA production blocks myogenic differentiation completely whereas all three peptides can release the CEA-imposed differentiation block;

FIG. 12 illustrates that substitution of the normal trans-membrane domain of the BGPa molecule (Tm) with the GPI membrane domain of CEA converts BGPa from a molecule that has no effect on myogenic differentiation (upper micrograph) to one (denoted BC-2) that inhibits myogenic differentiation completely (lower micrograph); and

FIG. 13 illustrates that substitution of the carboxy-terminal GPI-determining domain of NCAM-125 with the GPI domain of CEA giving the hybrid construct, NCAM 125-CEA, converts NCAM from a molecule that has no effect on myogenic differentiation to one that inhibits myogenic differentiation completely.

### DETAILED DESCRIPTION OF THE INVENTION

Although CEA had been previously considered as an inert marker of tumorigenicity, we suggested (Benchimol et al, 1989) that inappropriate CEA/NCA expression in cells still capable of proliferation could cause a distortion of tissue architecture and an inhibition of terminal differentiation which normally removes cells from the pool of cells with potential to proliferate, thus contributing directly to malignant progression.

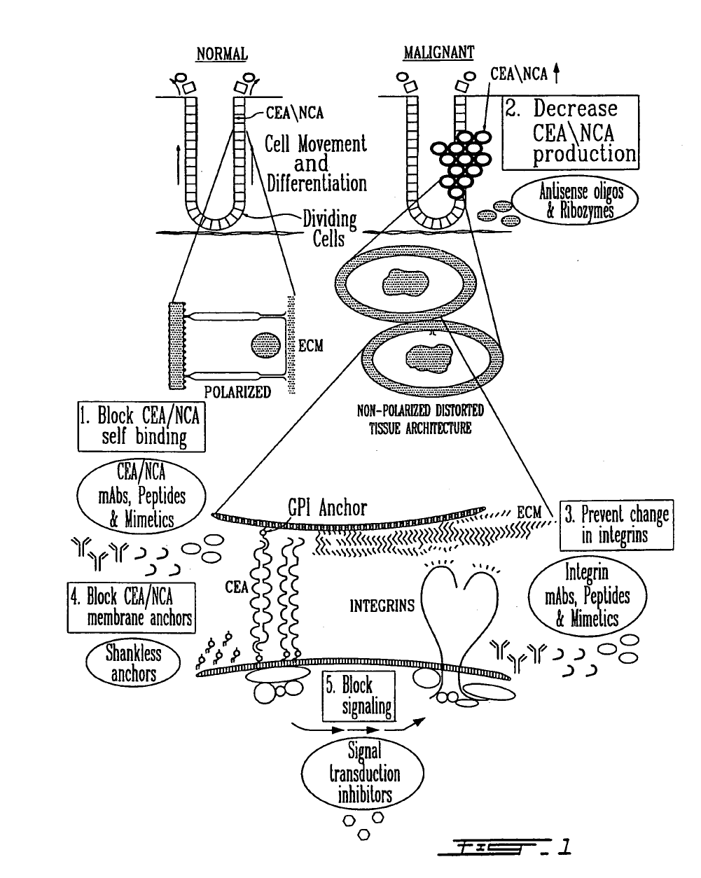

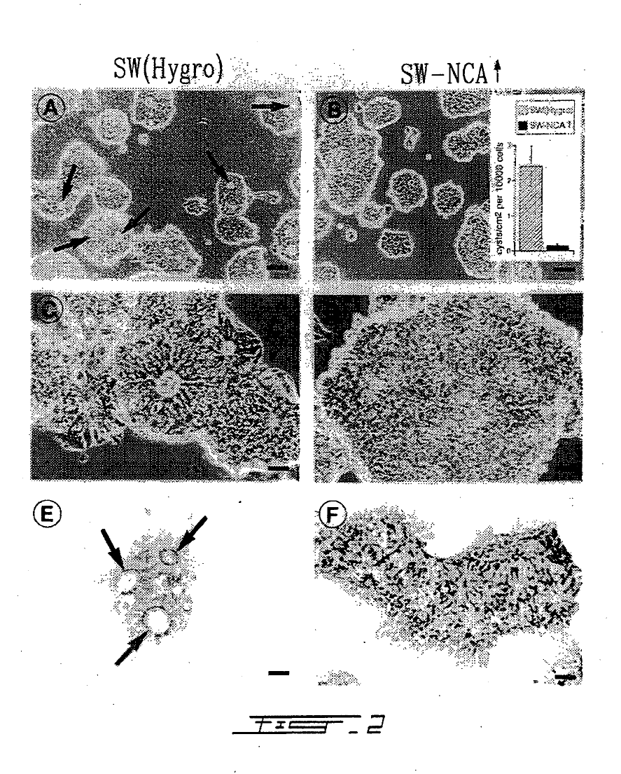

FIG. 1 illustrates the effect of CEA/NCA in normal cells which contributes to the development of a malignant tumor and the different sites of action of the routes 1, 2, 3, 4 and 5 of the present invention as described above.

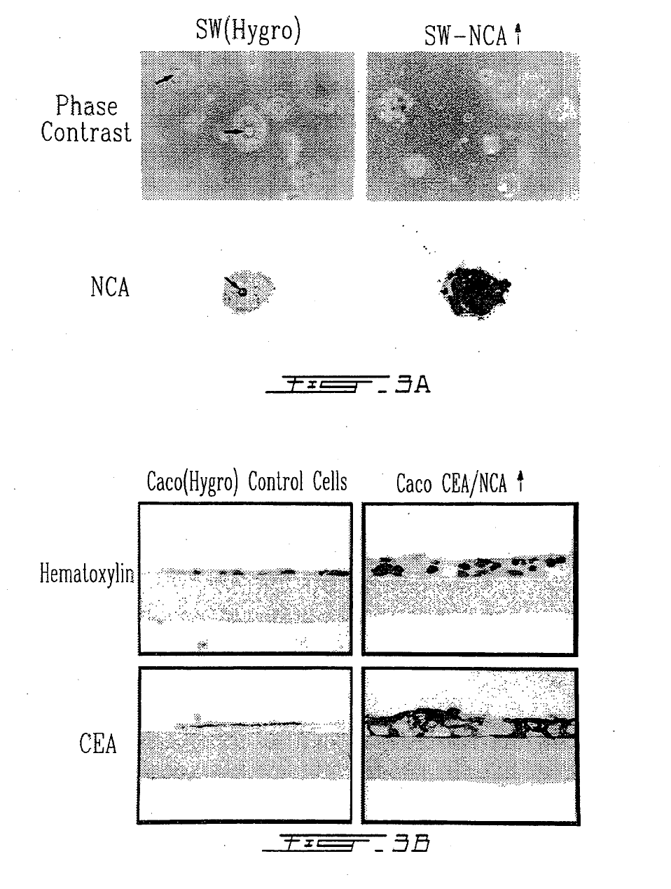

The above hypothesis was tested in several model systems: CEA expression in rat myoblasts (by transfection with CEA cDNA) was shown to block terminal myogenic differentiation completely (Eidelman et al, J. Cell Biol. 123:467-475, 1993); NCA had the same effect whereas a CEA family member that is down-regulated in cancer (BGP) had no effect (Rojas et al, Cell Growth Differ. 7:655-662, 1996); CEA expression inhibits terminal adipogenic differentiation of mouse adipocytes; CEA and NCA, but not BGP, expression inhibits the neuronal differentiation of mouse embryonal carcinoma cells; finally, human colorectal carcinoma cell lines, SW-1222 and Caco-2, that retain differentiation and polarization capacity, when forced by transfection to over-express CEA and NCA, lose their ability to form colonic crypt-like glandular structures with central lumens in both monolayer and spheroid culture and lose their ability to polarize, closely resembling the more progressed human colorectal carcinomas removed from patients (FIGS. 2, 3 and 4). Human colorectal carcinoma cell line SW-1222, stably transfected with empty expression vector [SW(Hygro)] or with vector containing NCA cDNA and thereby overexpressing NCA by 5-10 fold (in proliferating cells) [SW-NCA↑], grown in monolayer culture show lumens with radially arranged polarized cells in the case of the control SW (Hygro) cells but not in the case of the SW-NCA↑ cells (top, FIG. 2). Polarization of the control SW (hygro) cells is shown by the presence of rings of villin staining localized with microvilli at the lumens (bottom, FIG. 2). Thus the residual degree of glandular differentiation exhibited by the human SW-1222 colonocytes in monolayer is completely inhibited by over-expression of NCA (FIG. 2, inset).

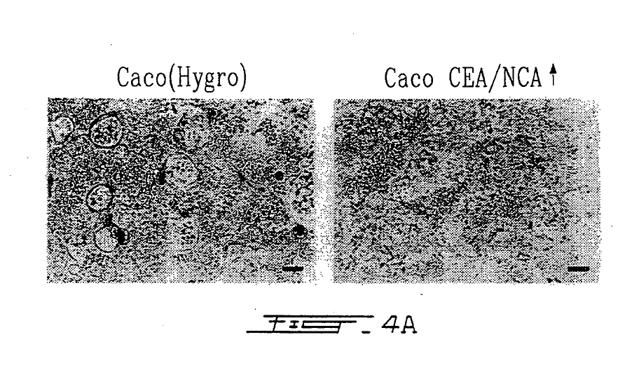

In FIG. 3 (top), SW-1222 (Hygro) cells grown in suspension in collagen gels form glandular spheroids consisting of radially arranged polarized colonocytes with central lumens selectively stained with anti-NCA mAb, whereas SW-NCA↑ cells form only irregular non-polarized cell masses with generalized NCA staining and no lumens. In FIG. 3 (bottom), human colorectal carcinoma cell line Caco-2, stably transfected with empty expression vector [Caco(Hygro)] or with vectors containing CEA cDNA and NCA cDNA [Caco CEA/NCA↑] and thereby overexpressing CEA & NCA by 20 fold (in proliferating cells), were cultured for 17 days on solid support. Cultures were sectioned vertically and stained with hematoxylin to show cell nuclei or with anti-CEA mAb to show the polarized expression of CEA at the apical upper surfaces of the monolayer of columnar colonocytes. Note the multilayered (stratified) configuration with circumferential CEA staining of the Caco CEA/NCA↑ cells. Thus CEA/NCA over-expression destroys the normal monolayered architecture of polarized columnar colonocytes of Caco-2, giving a tissue architecture closely mimicking that of colon carcinomas.

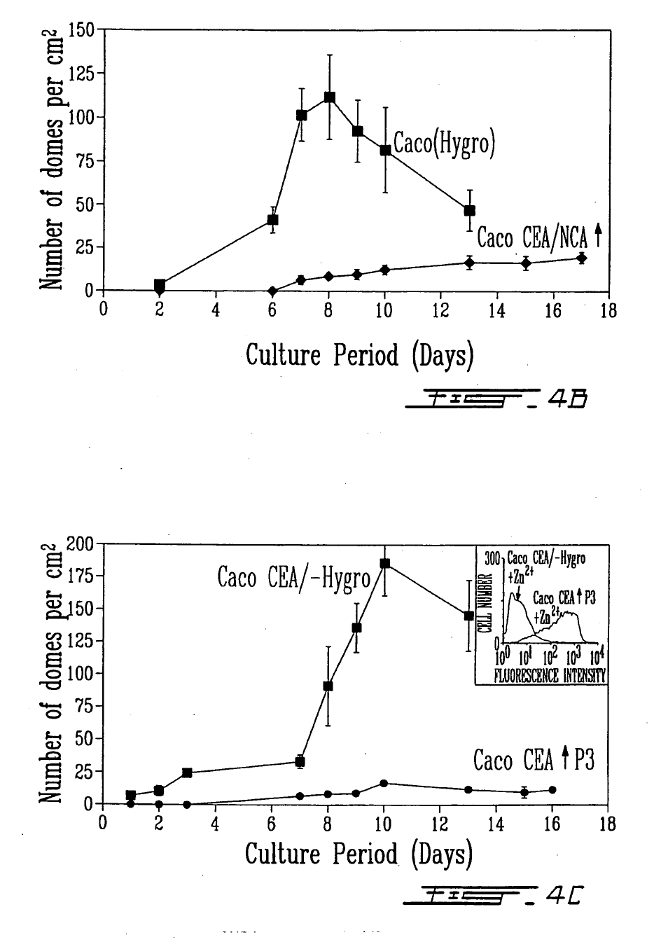

In FIG. 4B, dome formation, due to vectorial transport of solvent from apical to basolateral surfaces of colonocytes and an indicator of polarization, is strongly inhibited by over-expression of CEA/NCA in Caco-2 colonocytes. High levels of CEA expressed in Caco-2 cells transfected with CEA cDNA alone, Caco-CEA↑P3, also prevented the formation of domes. These cells, after culture in the absence of the selective agent (Hygromycin) resulting in loss of all CEA over-expression (see inset), renamed Caco-CEA-Hygro, dramatically regain their ability to form domes (FIG. 4C). Domes can be seen as raised circular areas of cells in the photomicrograph of control Caco (Hygro) cells (FIG. 4A, upper left) but not in Caco CEA/NCA↑ cells (FIG. 4A, upper right).

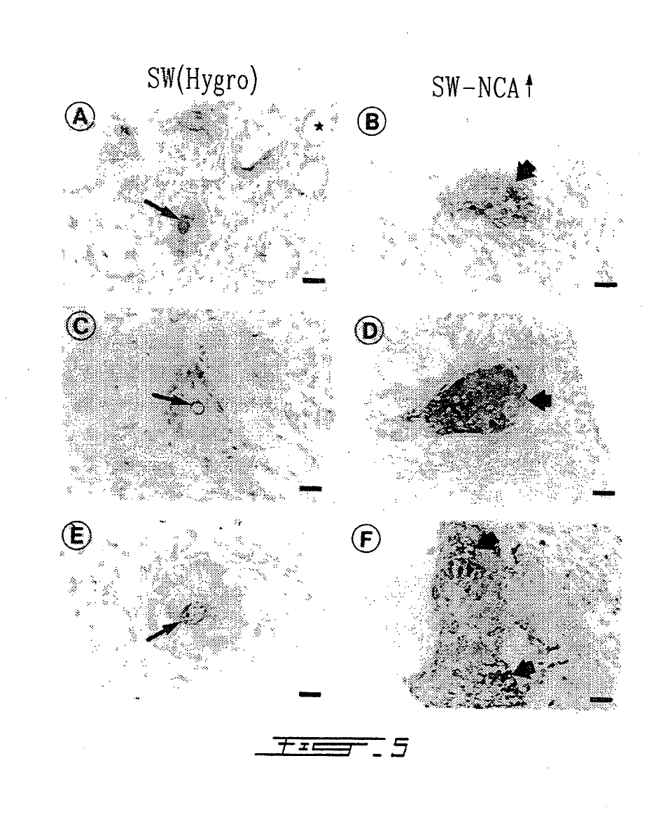

In an assay, developed in our laboratory, that tests the ability of human colonocytes to conform to normal tissue architecture (Ilantzis & Stanners, In Vitro Cell. Dev. Biol.—Animal 33: 50-61, 1997), over-expression of NCA causes a marked deterioration of their capacity to form colonic crypts (FIG. 5). Sections of minicolons obtained from growth of mixed aggregates of normal fetal rat colonocytes and 1% SW (Hygro) or SW-NCA↑ cells for 7-10 days under the kidney capsule of the nude mouse are shown in FIG. 5. The SW-1222 cells were identified by staining with an anti-CEA mAb. Note the dramatic deterioration of the quasi-normal tissue architecture of the SW (Hygro) cells as exemplified by rings of staining that are absent in the SW-NCA↑ cells over-expressing NCA.

In fact, we have shown that the cell surface levels of CEA and NCA on purified colonocytes from freshly excised colon carcinomas and adjacent normal tissue from patients, measured by FACS analysis, are markedly higher on the tumor cells relative to the normal cells and inversely correlated with the degree of differentiation of the tumors (Ilantzis et al, Lab Invest. 76(5):703-716, 1997); this represents clinical evidence in support of our hypothesis. Even microadenomas, the early precursors of colonic carcinomas showed upregulation of CEA and NCA in direct relation to their degree of dysplasia (Ilantzis et al, 1997).

In agreement with the suggestion that these effects of CEA/NCA over-expression could drive malignant progression, both the rat myoblasts (Screaton et al, J. Cell Biol. 137:939-952, 1997) and human CaCo-2 colonocytes transfected with CEA/NCA, but not with the empty transfection vector, produced tumors in nude mice with a strikingly reduced latent period.

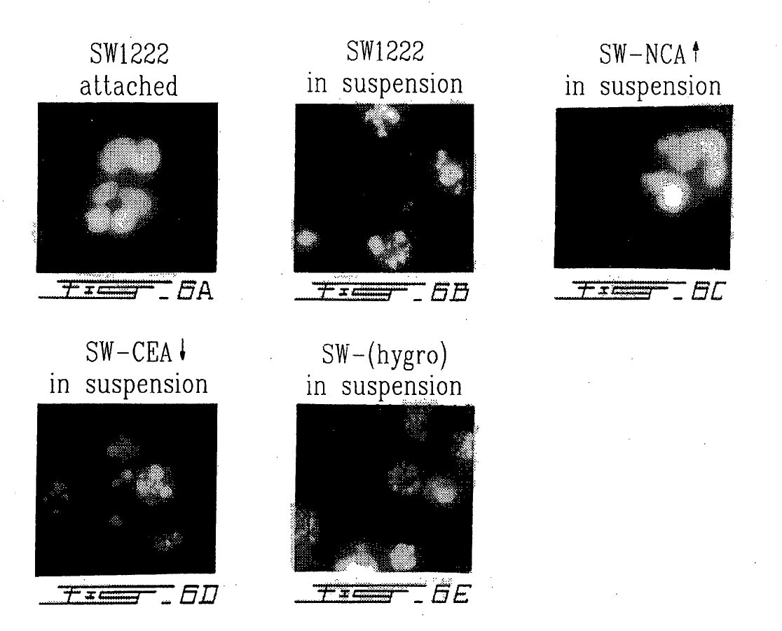

The above results support the contention that CEA and NCA are general inhibitors of terminal cellular differentiation. We have evidence that they achieve this by interference with the function of integrins responsible for cell/extracellular matrix interactions. The latter interactions are known to be required for many different types of cellular differentiation. Furthermore, we have direct evidence that these CEA/NCA-induced perturbations in integrin function inhibit anoikis, the apoptotic process that is employed to destroy cells that do not conform to normal tissue architecture (FIG. 6). CEA/NCA over-expression inhibits anoikis. SW-1222 cells attached to a solid support, stained with DAPI to show nuclear morhology, demonstrate whole nuclei (upper left). When incubated in suspension, parental SW-1222 cells, SW (hygro) cells and SW-CEA↓ cells (with CEA expression down-regulated) all show fragmented nuclei, indicative of anoikis (apoptosis). SW-NCA↑ cells, expressing much higher levels of NCA, show whole nuclei when incubated in suspension, thus not demonstrating anoikis. The overproduction of CEA/NCA affects the function of two particular integrins, α₅β₁ and αᵥβ₃; monoclonal antibodies directed against these integrins will reverse the inhibition of anoikis mediated by CEA/NCA.

The inhibition of apoptosis by the over-production of CEA/NCA is an alteration that will contribute to the development of cancer. It will also lead to resistance to cell killing by cytotoxic agents used in chemotherapy, which our results with certain such agents indicates. Thus, inhibition of these effects of CEA/NCA will lead to increased sensitivity to cytotoxic chemotherapeutic drugs.

The overall picture, then, is one of CEA/NCA-induced inhibition of terminal cellular differentiation, cellular polarization and anoikis, and an accompanying loss of tissue architecture. The net effect of these CEA/NCA-induced cellular changes is to promote malignancy and to produce resistance to cell killing chemotherapeutic agents.

1. Antibodies, Peptides and Mimetics:

The myogenic differentiation-blocking activity of CEA, at least, can be reversed by interference with the adhesion domains of CEA (the N and A3B3 domains—Zhou et al, J. Cell Biol. 122: 951-960, 1993) using domain-specific peptides made in bacteria or by a deletion in the N-terminal domain (Eidelman et al, J. Cell Biol. 123:467-475, 1993), indicating the necessity of CEA-CEA interaction for the effect. In fact, the ΔNCEA deletion mutant that is defective in its ability to effect a myogenic differentiation block can be potentiated by the application of cross-linking monoclonal antibodies. We therefore presume that it is CEA-CEA binding leading to clustering on the cell surface that is required for the differentiation block. Further work has shown that the glycophosphatidyl inositol (GPI) membrane anchor of CEA is also required for the myogenic differentiation block.

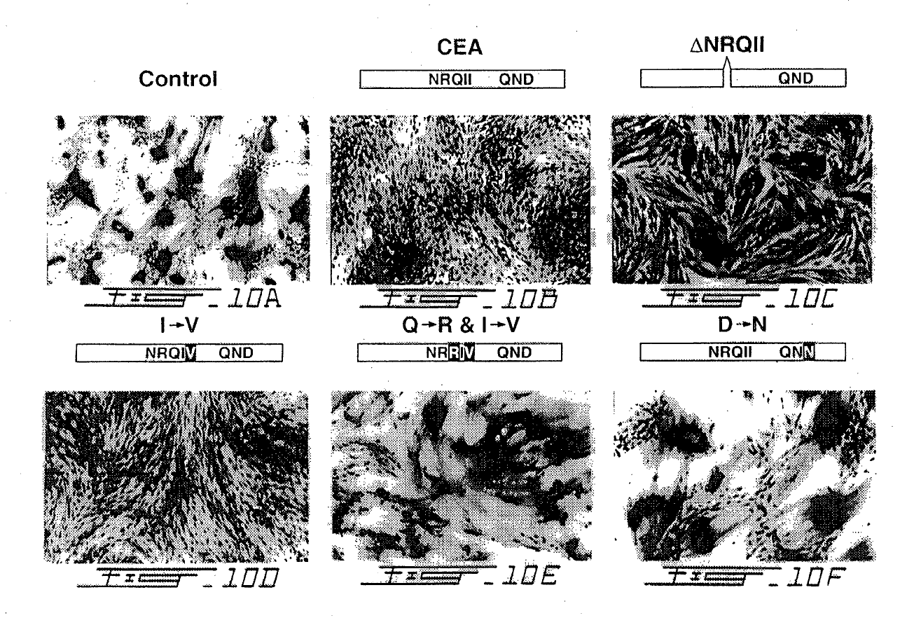

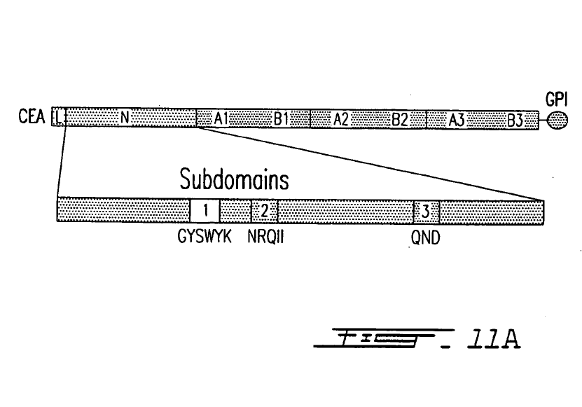

The precise subdomains in the N domain of the CEA molecule responsible for intercellular adhesion and for the myogenic differentiation block (and, by implication, other types of differentiation block) have been identified. These are NRQII, starting at amino acid #42 in the N domain of CEA (where the numbering begins at the first amino acid of the mature protein) which, when deleted or mutated to NRRIV (Q44R&I46V) or DRQII (N42D), abrogate both intercellular adhesion in transfected CHO-derived cells and the myogenic differentiation block; in addition, mutations giving amino acid substitutions at QND, starting at amino acid #80 in the N domain, giving QAD (N81A) or QNN (D82N) completely remove the ability of CEA to block myogenic differentiation without affecting its ability to mediate intercellular adhesion in CHO-derived cells (FIGS. 9 and 10). Mutations in a third subdomain, GYSWYK, starting at amino acid #30 in the N domain of CEA, also can remove the ability of CEA to block myogenic differentiation. The subdomains in the NH₂-terminal N domain of CEA that are required for intercellular adhesion of stable transfectants of CHO-derived LR cells and for the myogenic differentiation block of stable transfectants of rat L6 myoblasts are shown in FIG. 9. The positions of the 3 subdomains in the N domain of CEA that are required for adhesion and differentiation block are shown (top, FIG. 9). The effects of mutations in these subdomains on CEA-mediated adhesion, indicated by the % of cells remaining as single cells after incubation in suspension for 2 hrs, and on the CEA-mediated myogenic differentiation block, indicated by the % of nuclei in fused cells, are shown (bottom, FIG. 9). Photomicrographs of L6 transfectants indicated in FIG. 9, showing the extent of myogenic differentiation in some of the subdomain mutants after growth under differentiation conditions are presented in FIG. 10. The control L6 cells transfected with vector alone, L6-Neo, show extensive differentiation, whereas L6 cells transfected with CEA cDNA (L6-CEA) show none. Deletion of NRQII and especially point muations at D82 and Q44+I46 show release of the CEA-imposed differentiation block.

Thus the adhesive and differentiation-blocking activities of CEA can be separated, allowing the possibility of precise interference with the differentiation-blocking activity.

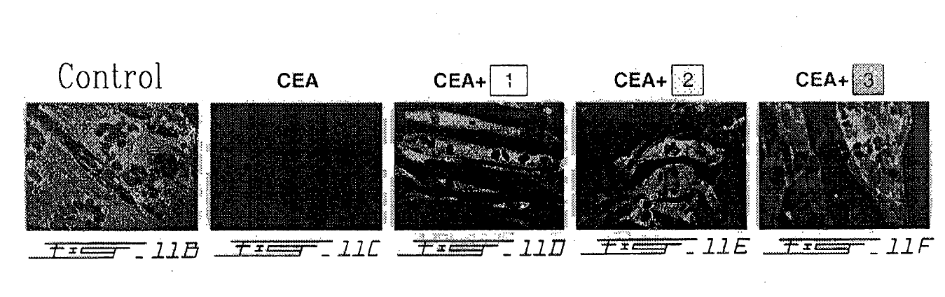

In fact, cyclic peptides including the sequences GYSWYK, NRQII and QND of the three subdomains of the CEA N domain have been applied to L6 myoblasts producing CEA and have dramatically released the CEA-imposed block in myogenic differentiation (FIG. 11). A similar release of the CEA-imposed myogenic differentiation block has been obtained by addition of monoclonal antibodies A20.12.2 (Zhou et al., Cancer Res. 53: 3817-3822, 1993) and others that we have shown to bind to an epitope including the YK residues of GYSWYK and the N residue of NRQII.

We thus propose to use peptides or mimetics representing these subdomains or monoclonal antibodies that bind to them that block the necessary CEA-CEA or NCA-NCA intermolecular interactions for the tumorigenic effects of CEA/NCA as agents for clinical use.

2. Antisense Agents:

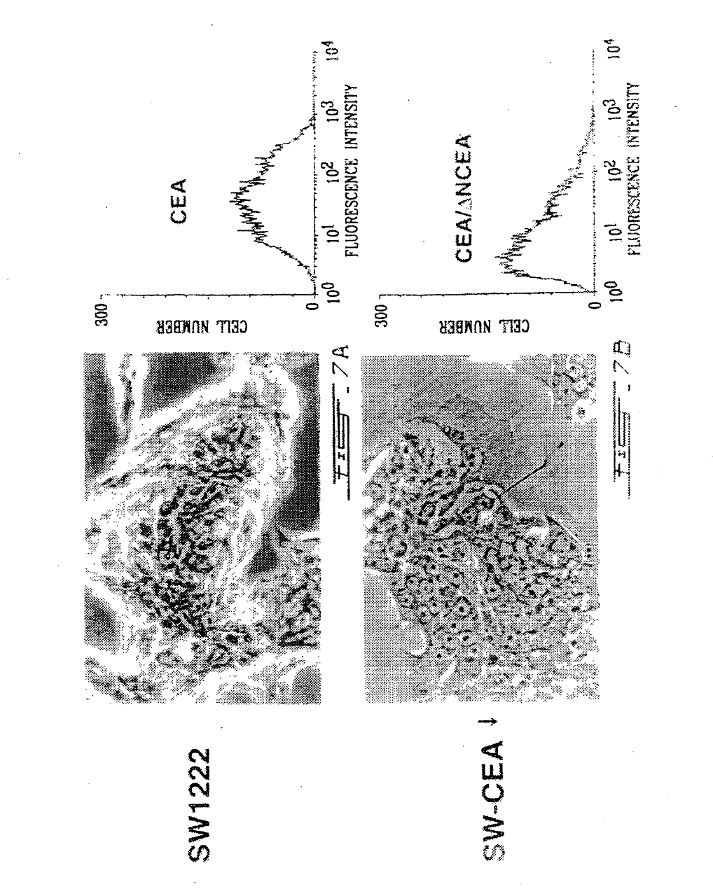

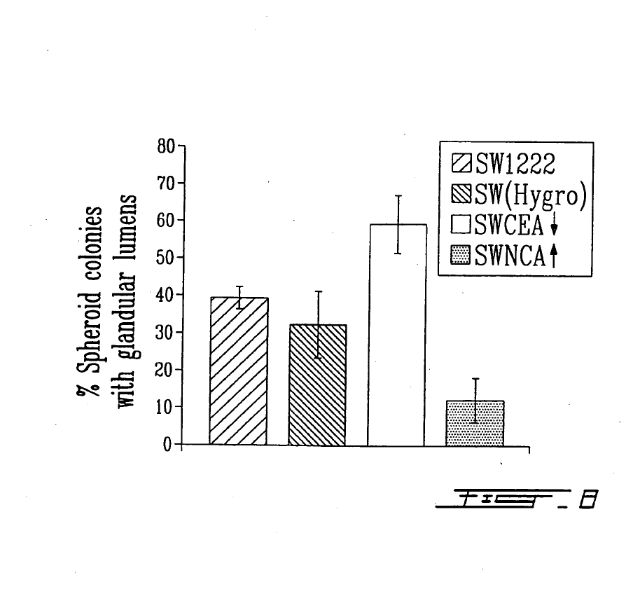

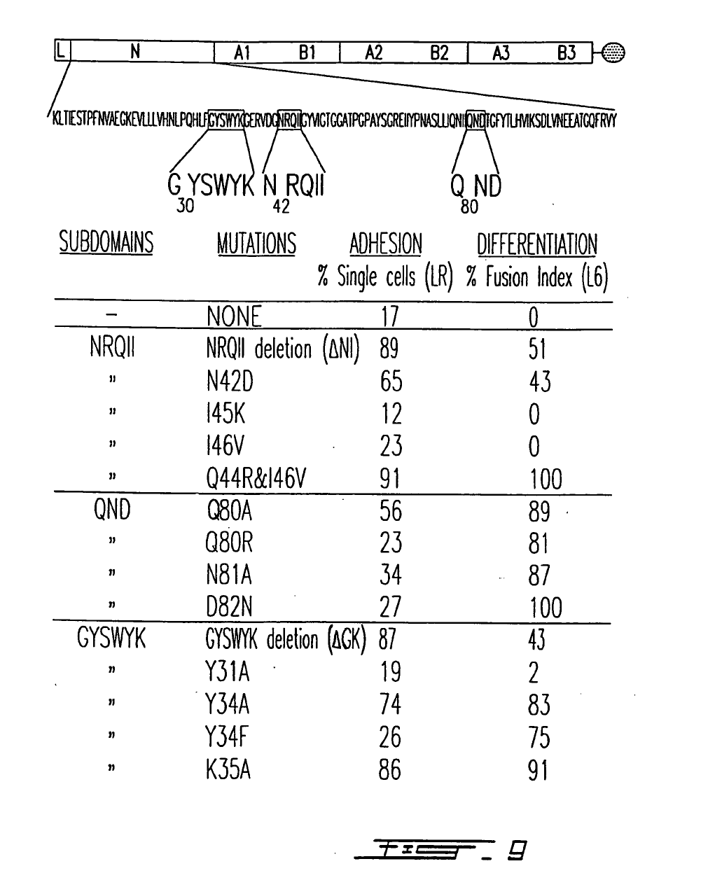

Importantly, human SW-1222 colon carcinoma cells transfected with a defective mutant of CEA and thereby actually producing less than normal levels of CEA, denoted SW-CEA↓, were more differentiated than the parental cells, in that they exhibited a more normal flat morphology in monolayer culture (FIG. 7) and more readily formed glandular spheroids in collagen gels (FIG. 8), thus indicating the possibility of reversal of the tumorigenicity of colonic carcinoma cells by forcing the down-regulation of CEA/NCA. Micrographs of control SW-1222 and SW-CEA↓ monolayer cultures are presented in FIG. 7, showing that down-regulation of CEA (see FACS profiles for cells stained with fluorescent CEA-specific mAb, D-14) results in a more normal, flatter morphology. The SW-CEA↓ line was obtained by stable transfection with the defective N-domain deletion mutant, ΔNCEA (Eidelman et al, 1993) (FIG. 7).

Such down regulation could be achieved by the application of CEA/NCA anti-sense oligonucleotides or anti-sense ribozymes to tumors or gene therapy with CEA/NCA antisense cDNA constructs.

3. Regulation of Integrin Function

We have recently shown that CEA/NCA over-expression inhibits terminal differentiation in so many different types of cells by perturbing a molecular process common to all, that of interaction with the extra-cellular matrix (ECM). The major class of cellular receptors responsible for ECM interactions are the integrins; integrin-ECM interactions are known to be involved in many types of differentiation, in the maintenance of tissue architecture and in anoikis (apoptosis). The particular integrin disturbed in its function by CEA/NCA expression (but not by BGP or other controls) is α₅β₁, as shown by reversal of the inhibitory effect of CEA/NCA on anoikis of suspended rat L6 myoblasts and human Caco-2 cells by a monoclonal antibody against this integrin. This mAb or peptides/mimetics that mimic its effects on α₅β₁ function could be administered to patients bearing CEA/NCA expressing tumors and would be expected to release the differentiation block imposed by CEA/NCA. In mouse P19 embryonal carcinoma cells, the integrin affected is αᵥβ₃. The agents described above in routes 1, 2, 3, 4 and 5 of the present invention should cause CEA/NCA over-expressing tumor cells to differentiate terminally, thus removing them as potential colonizing cells in the body.

4. Shankless Anchors:

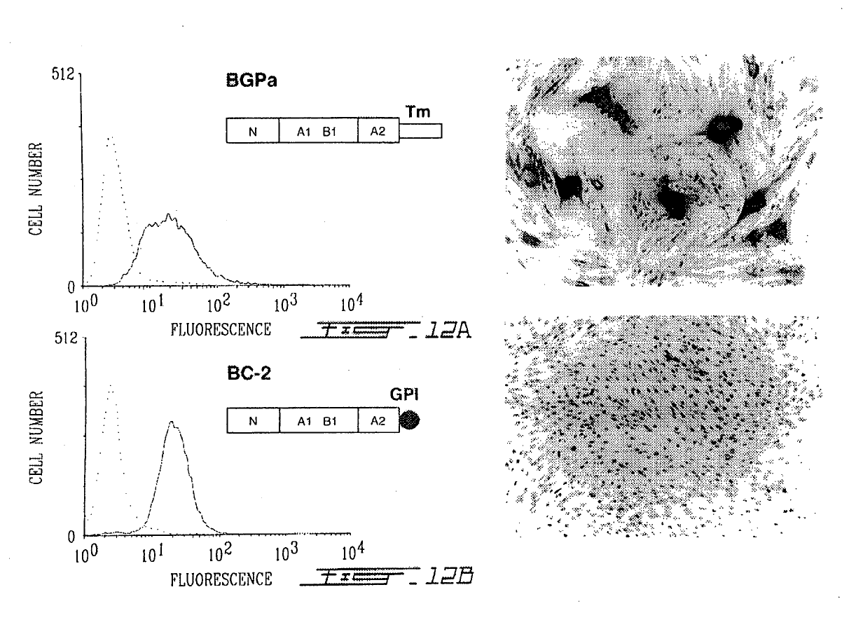

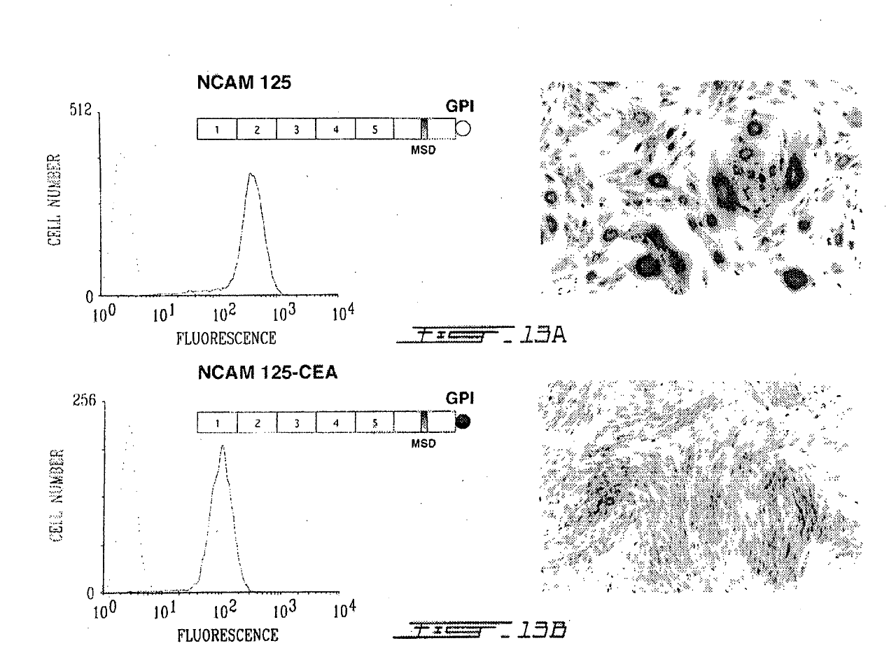

We have shown that the structural features of the CEA molecule required for the differentiation are as follows: first, external domains capable of self association and, second, attachment of these to the hydrophobic carboxy-terminal domain of CEA; the latter domain is normally cleaved during processing events resulting in the formation of a GPI membrane anchor. Thus the trans-membrane linked BGPa member of the CEA family, normally without effect on myogenic differentiation, can be converted to one that blocks differentiation by the addition of the CEA GPI domain (FIG. 12). Substitution of the normal trans-membrane domain of the BGPa molecule (Tm), including its cytoplasmic domain, with the GPI membrane domain of CEA (GPI) converts BGPa from a molecule that has no effect on myogenic differentiation (upper micrograph) to one (denoted BC-2) that inhibits myogenic differentiation completely (lower micrograph). FACS profiles showing cell surface levels of BGPa and BC-2 indicate equivalent levels of expression (FIG. 12). Conversely, the substitution of the GPI anchor of CEA with the transmembrane anchor of BGPa, converts CEA from a molecule that blocks myogenic differentiation into one that has no effect. Strikingly, the GPI membrane-linked NCAM splice isoform, NCAM-125, which has no effect on myogenic differentiation, can also be converted to a molecule with differentiation blocking capacity by the addition of the CEA GPI domain (FIG. 13). The GPI-linked NCAM (neural cell adhesion molecule) splice isoform with the muscle-specific domain (MSD), NCAM 125, even at relatively high levels of cell surface expression in rat L6 stable transfectants, has no effect on myogenic differentiation. Substitution of its own carboxy-terminal GPI-determining domain (open circle) with the GPI domain of CEA (full circle) giving the hybrid construct, NCAM 125-CEA, however, converts it into a molecule that inhibits myogenic differentiation completely (FIG. 13).

Strategy 1. depends on interference with the binding domains of CEA which are the self-binding domains naturally associated with the CEA GPI anchor. The present strategy is targeted to the GPI domain itself. CEA molecules lacking binding domains, consisting of the GPI anchor alone, with little or no attached peptide (“shankless anchors”), can be generated by enzymatic cleavage or by the use of CEA cDNA constructs with deleted binding domains. These can be applied directly to cells blocked in differentiation by CEA/NCA and, as has been shown for other GPI-linked molecules, should successfully embed themselves from the external milieu into the membrane of the cells. Inhibition with the differentiation-blocking activity of the endogenous CEA/NCA molecules is anticipated via competition by the CEA shankless anchors for elements of the molecular pathway required for the CEA/NCA effect. Release of the CEA-mediated myogenic differentiation block has, in fact, been observed by co-transfection with CEA cDNA constructs producing CEA molecules with intact GPI anchors but defective external binding domains.

The above inhibitory effect of GPI anchors could be applied in principle to inhibit the activity of any GPI-linked molecule. This extends the potential utility of the present invention to include many types of GPI-linked molecules with a wide range of biomedical effects.

Development of the Present Invention Over the Next Year

1. Antisense oligonucleotides, ribozymes and cDNA constructs will be prepared and tested for their ability to reduce the expression of CEA/NCA in SW-1222 (NCA↑) cells and Caco-2 (CEA/NCA↑) cells. The effects on the cellular and tissue architecture and tumorigenicity of these transfectants so treated will be measured, expecting a reversal to more normal behaviour.
2. Peptide mimetics against the differentiation-blocking subdomains of CEA will be developed and tested for their ability to release the myogenic differentiation block and to reduce the tumorigenicity of L6 myoblast transfectants expressing CEA. The mimetics will also be tested for ability to restore normal cell and tissue architecture and reduce tumorigenicity of SW-1222 (NCA↑) and Caco-2 (CEA/NCA↑) cells.
3. CEA shankless anchors will be prepared and applied to L6 myoblast transfectants expressing CEA, testing for their ability to release the CEA-imposed differentiation block.
4. The mAb against α₅β₁ will be tested for its ability to reverse all of the effects of CEA expression on the L6 myoblasts, including impairment of binding to ECM and the differentiation block. The test will be extended to SW-1222 (NCA↑) and Caco-2 (CEA/NCA↑), looking for restoration of normal cell and tissue architecture.

The present invention will be more readily understood by referring to the following example which is given to illustrate the invention rather than to limit its scope.

### EXAMPLE I

### Peptide/Mimetic, Monoclonal Antibody or Drug Screening Assay

L6 rat myoblasts transfected with CEA/NCA cDNA and thereby blocked in their differentiation provide the most sensitive assay for screening agents capable of releasing the differentiation block.
1. L6 (CEA/NCA) cells producing CEA or NCA are seeded into tissue culture plates containing multiple wells and cultured until forming a confluent monolayer.
2. The medium is changed to a medium poor in growth factors (DMEM plus 2% horse serum) that stimulates differentiation. At the same time the agent to be tested is added at a series of concentrations to an appropriate number of cultures in the wells.
3. The culture plate is incubated for 5-7 days. If the agent being tested is unstable, additional agent is added during this incubation period.
4. At the end of the incubation period, the medium is removed and the cultures stained with hematoxylin. Release of the CEA/NCA-imposed differentiation block is easily assessed by the presence of mutinucleated giant cells that also stain positively with anti-myosin antibody (see FIGS. 10 and 11) and quantitated by the percentage of total nuclei in cells with >3 nuclei.
5. Agents that give high levels of myogenic differentiation are then tested for effects on human colonocytes aberrantly expressing high levels of CEA/NCA. The simplest assays are the test for formation of glandular structures of polarized cells by SW-1222 (NCA↑) cells in monolayer (see FIG. 2) and the assay for dome formation and polarization of Caco-2 (CEA-NCA↑) cells (see FIG. 4).
6. Agents that reverse the CEA/NCA-mediated inhibition of cellular polarization, tissue architecture and differentiation of human colorectal carcinoma cell lines are then tested for their ability to inhibit the tumorigenicity, the formation of metastases and reverse the undifferentiated characteristics of the same cell lines injected into the cecum or spleen of nude mice. They are also tested for their efficacy in restoring normal tissue architecture in our mouse tissue architecture assay (Ilantzis & Stanners, In Vitro Cell. Dev. Biol.—Animal 33: 50-61, 1997). Finally, successful agents are administered to patients bearing cancers that over-produce CEA/NCA, with the expectation that they will block the growth of tumors and their derived metastases by forcing them to differentiate.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method of relieving a CEA/NCA-imposed inhibition of differentiation and/or apoptosis comprising an incubation of primary or secondary tumour cells with an agent which disrupts one of an interaction between CEA/NCA subdomains having sequences selected from G₃₀YSWYK, N₄₂RQII, Q₈₀ND, and a functional interaction between said subdomains and integrin α₅β₁ and αᵥβ₃.

2. A method for selecting a peptide or peptide-derived mimetics which can modulate a differentiation-blocking activity associated with a subdomain of CEA/NCA in a malignant tumor, wherein said subdomain is selected from the group consisting of sequences G₃₀YSWYK; N₄₂RQII; Q₈₀ND; sequences including epitopes of 3 to 6 amino acids in the N-terminal 107 amino acid domain; and sequences including epitopes of 3 to 6 amino acids in the internal A3B3 178 amino acid domain of CEA, wherein said peptide or peptide-derived mimetics is selected as a modulator of said differentiation-blocking activity, when a tumor cell incubated with said peptide or peptide-derived mimetics, displays a significantly modified differentiation status compared to a tumor cell incubated in the absence thereof.

3. Peptides and/or peptide-derived mimetics obtained by the method of claim 2, wherein said peptide-derived mimetics interacting with subdomains of CEA/NCA involved in the differentiation-blocking activity associated with malignant tumors, wherein said subdomains are selected from the group consisting of sequences G₃₀YSWYK, N₄₂RQII, and Q₈₀ND.
